(19) United States
(12) Patent Application Publication
(10) Pub. No.: US 2005/0080651 A1
(43) Pub. Date: Apr. 14, 2005

(54) System and method for remote processing of pharmacy orders

(76) Inventors: Kelly L. Morrison, Sugar Land, TX (US); Ronald Wayne Graham, Katy, TX (US); Kent Black, Katy, TX (US)

Correspondence Address: CARDINAL HEALTH, 7000 CARDINAL PLACE, LEGAL DEPARTMENT - INTELLECTUAL PROPERTY, DUBLIN, OH, 43017, US

(21) Appl. No.: 10/686,385

(22) Filed: Oct. 14, 2003

(51) Int. Cl. G06F 17/60

(52) U.S. Cl. 705/2

(57) ABSTRACT

A system and method for remote pharmacy order processing is described. Orders from hospitals are transmitted to a site for centralized order queue management. Each order is identified and added to a queue for the originating hospital. Orders are reviewed and authorized at remote order processing centers by licensed pharmacy personnel. Computers at the remote order processing centers are linked to hospital pharmacy information systems. A pharmacist at a remote order processing center selects a hospital, reviews orders from the queue for the selected hospital, and enters them directly into the hospital's pharmacy information system. The present invention also supports service level tracking and alerts for aging orders. Commitments are specified in service level agreements. The center then processes the orders according to the commitments. Hospital specific clinical initiatives and policies are stored in a central data repository and made available to personnel to ensure hospital policy compliance.

### FIELD OF THE INVENTION

The present invention relates generally to the field of tele-pharmacy in which pharmacy orders are reviewed and authorized at remote pharmacy facilities. Specifically, the present invention is a system and method for remote processing of pharmacy orders in which pharmacy personnel at remote pharmacy facilities access pharmacy information systems of multiple healthcare facilities to review and authorize their pharmacy orders.

### BACKGROUND OF THE INVENTION

Approximately 4,000 hospitals in the U.S. do not have twenty-four (24) hour pharmacy services to provide review and authorization of pharmacy orders. However, physicians in these same hospitals are writing pharmacy orders to prescribe medications twenty-four hours each day. As a result, nurses often administer the medications prior to a pharmacist's review and authorization of an order, or they wait to administer the medications until the pharmacy service resumes the next day and the order is reviewed and authorized. This practice results in issues of medication safety for patients.

In many instances, the pharmacy orders are reviewed retrospectively (i.e., after administration of the drug to a patient) by a pharmacist on site at the hospital. If the hospital pharmacy information system is linked to a system for automated and authorized release of drugs to patients, a nurse must override the automation system in order to receive the drug, or refrain from dispensing and administering the drug until the pharmacy opens the following day. In any case, the practice of retrospectively reviewing and authorizing orders increases the potential for increased medication errors, allergies, and drug interactions. The problem is nationwide and contributes to the increasing national awareness of medication errors.

The reason that many hospitals do not provide twenty-four hour pharmacy service is that there is a national shortage of pharmacists. Hospitals are trying to find more pharmacists in an extremely tight labor market and consequently, are paying higher salaries which increase costs. Even if the hospital can find pharmacists, simply providing more hours of pharmacy coverage further increases overhead and operating costs for hospitals. With the short supply of pharmacists and the resulting costs of operating a pharmacy twenty-four hours a day, the demand for hospitals to provide twenty-four hour coverage of the pharmacy cannot be met. Furthermore, providing pharmacist coverage 24/7 in all hospitals will be cost prohibitive based on the current hospital reimbursement for pharmacy services.

The field of tele-pharmacy has started to develop only in recent years. In current systems, pharmacists at a remote center are able to access only the same pharmacy information system installed at multiple hospitals within a particular healthcare system on the same wide area network or multiple hospitals each with its own pharmacy information system. Further, the existing field utilizes a manual process to retrieve faxed orders. In current systems, as illustrated in FIG. 1, nurses at hospitals fax the drug orders to a physical fax machine located at a remote center. Referring to FIG. 1, an order 100 is faxed from a nursing station fax machine 102 at a hospital and is received at a fax machine 104 at a remote center. A pharmacist 110 at the remote center retrieves the order 106 from the fax machine 104. The pharmacist 110 then connects to the hospital's pharmacy information system 114 from a workstation 108 at the remote center. The connection between the remote center workstation 108 and the hospital's pharmacy information system 114 may be established via the Internet 112. Once connected to the hospital's pharmacy information system 114, the pharmacist 110 may enter and review orders on the hospital's pharmacy information system 114.

None of the existing systems centralize and automate multiple hospitals' pharmacy orders or provide multiple order processing centers with specific hospital queue identification and tracking. Therefore, there is a need for a system and method that allows pharmacy personnel at remote centers to process pharmacy orders for multiple hospitals through a centralized system.

### SUMMARY OF THE INVENTION

The present invention is a remote order processing system and method comprising remote order processing centers that provide hospital pharmacies with supplemental resources to facilitate timely and efficient review and authorization of all pharmacy orders. Each remote order processing center is a licensed pharmacy staffed by licensed/registered pharmacists and certified/registered pharmacy technicians. The remote order processing centers provide seamless order processing service by linking their computers directly to pharmacy information systems at hospitals and emulating those systems. Using technology such as a virtual private network, dial up connections, high-resolution fax servers with archiving capability, scanners and other technologies, the pharmacy orders are transmitted (via fax, email, or scanner) for centralized queue management, and then are accessed via a secure connection at the remote order processing centers for processing by pharmacists. A pharmacist at a remote order processing center may view an electronic version of the order and enter it directly into the hospital's pharmacy information system. The pharmacist functions as if physically on-site at the hospital. The hospital's pharmacy information system may be linked to profile driven automation and authorization of the orders. If present, the automation system releases the approved medication for administration to the patient. Nurses at remote hospital facilities dispense medications based on pharmacy orders that have been reviewed and authorized by a pharmacist prior to being dispensed to a patient.

The present invention supports remote pharmacy order entry into different pharmacy information systems for multiple hospitals concurrently. When processing pharmacy orders for multiple hospitals using different pharmacy information systems, it is desirable for all hospital pharmacy orders to be sent to a site for centralized order queue management that identifies each order sheet by the hospital and nursing station where the order originated. Order sheets according to the present invention are identified by hospital in several ways including the specific CSID, ANI or DNIS number for faxed orders, sender email address for emailed orders, or unique hospital identifiers that are programmatically assigned to scanned orders. The invention integrates fax servers and document management polling and storage applications to electronically capture faxed, emailed, and scanned pharmacy orders and convert them to an electronic image in order to be queued (e.g., using a “first in—first out” process) according to the hospital that originated the order.

In addition to providing master electronic hospital queues, the present invention also supports hospital service level tracking and alerts for aging orders and provides operational and clinical metrics related to order volume, processing time and clinical consultation activity per hospital facility. Each hospital's service requirements may be stated in an agreement that specifies service commitments to be met by a remote processing center (e.g., processing of orders within a specified time period). The remote processing center to which the hospital's orders are directed then processes the orders according to service level commitments that are communicated to personnel using features of the present invention.

The remote order processing system also provides hospitals with the option of transferring responsibility for administrative functions related to remote order review and authorization. Hospitals can utilize the service during evening hours when the pharmacy is closed, during peak periods of demand, during periods when access to pharmacy personnel is limited, or as a means of processing the majority of orders. The distinct advantage of the remote order processing system is the prospective and concurrent review of the patient's medication order to prevent potential medication errors.

The present invention provides an innovative and unique alternative to hospitals that are affected by changing environmental forces. Some of these forces are the pharmacist shortage, increased focus on medication safety, increasing accreditation standards, and rising pharmacy costs and declining hospital profitability. Responding to these changing environmental forces and continuing to deliver services under their current delivery model would be very difficult and cost prohibitive. The unique remote order processing system and method of the present invention offers the innovative alternative to address these issues.

### BRIEF DESCRIPTION OF THE DRAWINGS

FIG. 1 is a diagram of a prior art faxing process for pharmacy orders;

FIG. 2 is a diagram of a centralized order queue management process according to an example embodiment of the present invention;

FIG. 3 is a diagram of the technology process and flow that creates and maintains the centralized client order queues for an example embodiment of the present invention;

FIG. 4 is a diagram of a process for remote authorization of an automated medication dispensing system for an example embodiment of the present invention;

FIG. 5 is a diagram of a remote order processing standard station configuration for an example embodiment of the present invention;

FIG. 6 is a diagram of a remote order processing station workflow for viewing a consolidated queue for an example embodiment of the present invention;

FIG. 7 is a diagram of a remote order processing station workflow for accessing specific hospital orders for an example embodiment of the present invention;

FIG. 8 is a diagram of a remote order processing station workflow for accessing a specific order for an example embodiment of the present invention;

FIG. 9 is a diagram of a remote order processing station workflow for accessing specific hospital orders for hospitals with internal order management systems for an example embodiment of the present invention;

FIG. 10 is a diagram of a remote order processing station workflow for entering hospital orders using a hospital internal order management system for an example embodiment of the present invention;

FIG. 11 is a diagram illustrating remote order processing proprietary software applications for an example embodiment of the present invention;

FIG. 12 is a master hospital queue screen for an example embodiment of the present invention;

FIG. 13 is a hospital detail queue screen for an example embodiment of the present invention; and

FIG. 14 is an order view screen for an example embodiment of the present invention.

### DESCRIPTION OF EXAMPLE EMBODIMENTS

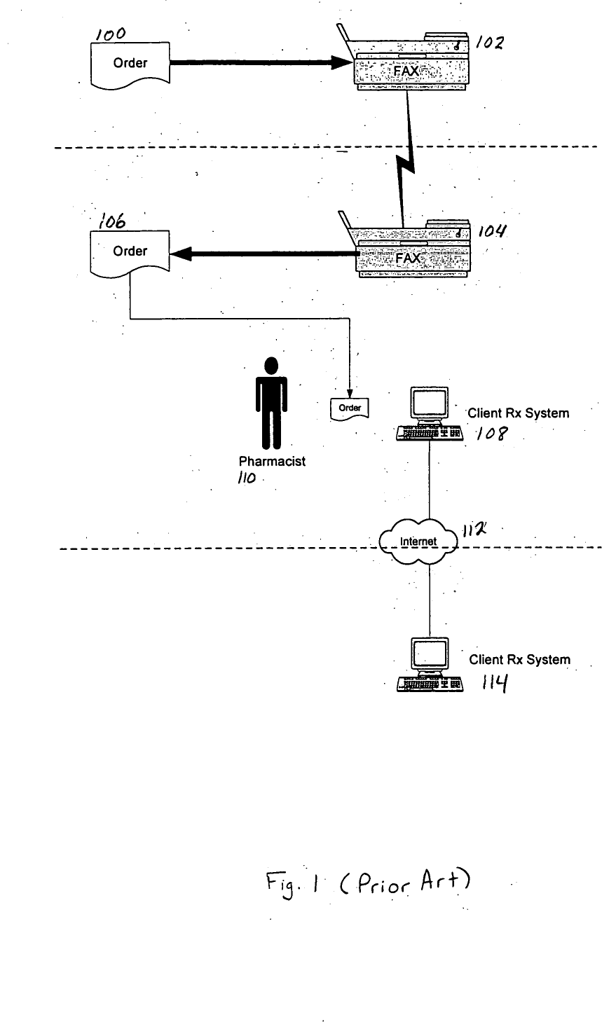

Remote order processing centers according to the present invention access over secure Internet connections multiple hospitals that have various different pharmacy information systems and various different types of automation to ensure that hospital specific pharmacy orders are processed, authorized, and returned to nursing staff within a specified timeframe (e.g., 60 minutes of receipt). Processing of orders according to the present invention involves pharmacy staff complete one or more of the following tasks: order review (review of order information), order entry (entry of order in a hospital pharmacy information system), order authorization (authorization for release of the medication to the patient). The remote order processing system and method of the present invention is described in FIGS. 1-14.

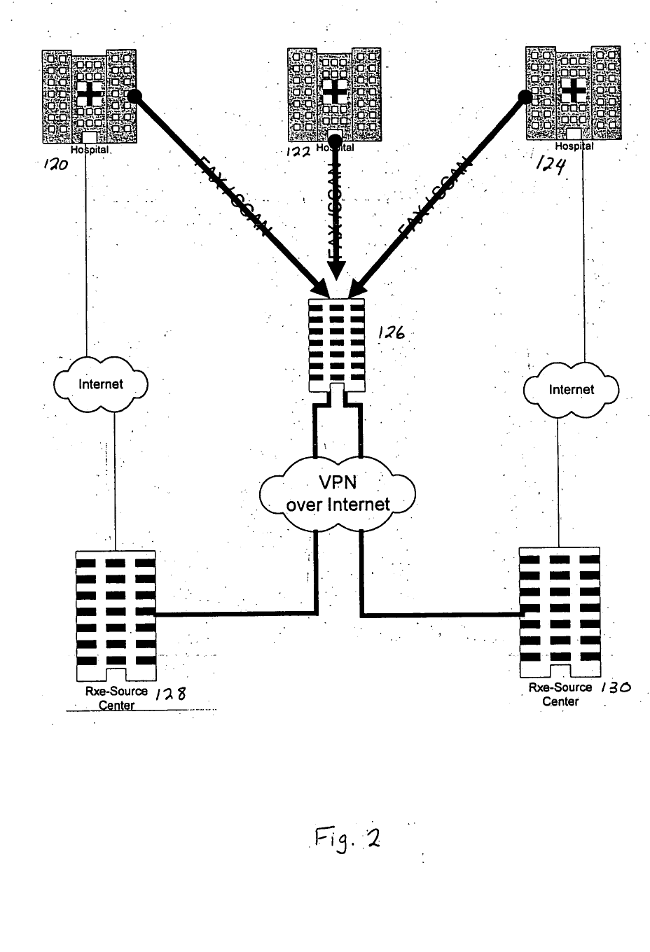

Referring to FIG. 2, a centralized order queue management process according to an example embodiment of the present invention is shown. The centralized order queue management process is the automated process by which remote order processing centers electronically receive hospitals' orders which may be faxed, scanned, emailed, etc. Orders are transmitted from a plurality of hospital pharmacy information systems 120, 122, 124 to a central order queuing site where they are received at or entered into a server 126 (e.g., a fax server, document server, etc.) and organized in hospital queues. A separate order queue is maintained for each hospital. The orders may be stored electronically in hospital specific directories.

If the server receiving the orders is a fax server, fax server software such as RightFax from Captaris may be used. This product receives and digitizes all hard copy faxed images. The digitized images are then transferred to a document management server that assigns order sheets to appropriate hospital queues based on CSID, ANI and/or DNIS information received from RightFax. Emailed orders are identified based on the sender email address. For scanned order, a unique hospital identifier may be assigned to the order. Whether faxed, emailed, or scanned, the queue management process identifies each order sheet by the hospital and nursing station where the order originated.

It is understood that the functions of the various servers described in accordance with FIG. 2 may be provided in one software server or distributed-among multiple software servers executing on one or more computers. As defined herein, the term “order server” refers to any single software server or combination of software servers (e.g., fax server, email server, or other order receiving server and document management server) that provide features and functionality for receiving orders, digitizing or producing images of orders, and organizing orders in queues.

Pharmacists at a plurality of remote order processing centers 128, 130 connect via a virtual private network over the Internet to a server 126 at the central order queuing site. Once connected to the server 126, they may view order queues for different hospitals. The pharmacists then use secure Internet connections (e.g., 128-bit encrypted) to access the hospitals' pharmacy information systems 120, 122, 124 and process orders.

Remote access to a hospital's pharmacy information system may be achieved using various methods. A first access method is remote controlling of pharmacy computers at the hospital's facility. A DSL or other high-speed connection may be established between a remote order processing center computer and a pharmacy computer to allow remote control of the pharmacy computer. Control via the Internet may be achieved using an Internet Service such as gotomypc.com™ from Expercity, Inc. Gotomypc.com software is installed on designated pharmacy computers located at hospital pharmacy facilities. Remote order processing center personnel access the Gotomypc.com website and enter designated user credentials for each hospital pharmacy to establish a secure connection between the remote order processing center computer and a pharmacy computer located at the hospital thus allowing remote order processing center personnel access to a remote hospital's pharmacy information system. Remote order processing center personnel accessing the hospital pharmacy information system may be provided with designated login accounts providing pharmacist equivalent rights.

A second access method is remote controlling pharmacy workstations via a direct network connection between the center network and workstations at the hospital's facility. A T1 line connection is used to connect the remote order processing center network to workstations at the hospital facility. The hospital provides its preferred commercial VPN client software for use on computers at the remote order-processing center. When the VPN software is launched from a remote order processing center computer and designated user credentials are entered, a secure connection or “virtual private network” (VPN) between the remote order processing center computer and a hospital computer is established. At that point, some form of remote control software such as PC Anywhere by Symantec is launched to provide to remote order processing center personnel the ability to remote control a workstation at the hospital facility that accesses the pharmacy information system.

A third access method may be accomplished by executing the hospital's pharmacy information system from the remote order processing center network. A T1 line connection may be used to connect the remote order processing center network and hospital pharmacy information system. The hospital provides a VPN client for use at the remote order processing center as well as any applicable pharmacy software licenses that may be needed to run the pharmacy information system. Remote order processing center personnel accessing the hospital pharmacy information system may be provided with designated login accounts providing pharmacist equivalent rights.

Finally, for web-based hospital pharmacy information systems, access may be obtained through the web. Using this method, pharmacy personnel need only designated login accounts providing pharmacist equivalent rights.

Once a secure connection between a remote order processing center computer and hospital pharmacy computer is established, remote order processing center personnel can access a hospital's pharmacy information system the following ways:

Use “Gotomypc.com” or PC Anywhere: These software products may be used to remotely control a computer located at the site of the hospital's pharmacy information system. Gotomypc.com provides a secure connection to a remote computer while allowing the remote computer to also be remote controlled by the computer that initiated the remote connection. PC Anywhere does not provide a remote connection to a computer, but provides remote control capabilities. Therefore, PC Anywhere is used in conjunction with another method such as “Gotomypc.com,” VPN, or dial up via modem to provide remote access to the computer.

Direct Network Connection: A direct network connection to the site running the hospital's pharmacy information system software may be established from a remote order processing center computer. Remote order processing center personnel may then access the hospital's pharmacy information system software to process orders.

Computers in each remote order-processing center share a high-speed connection (TI line) to the Internet to ensure adequate connectivity performance when remotely accessing hospital pharmacy information systems. Furthermore, to ensure redundancy in the event of a TI Internet connection failure, each center has a backup high-speed alternative such as DSL, cable modem, satellite, or a shadow TI line.

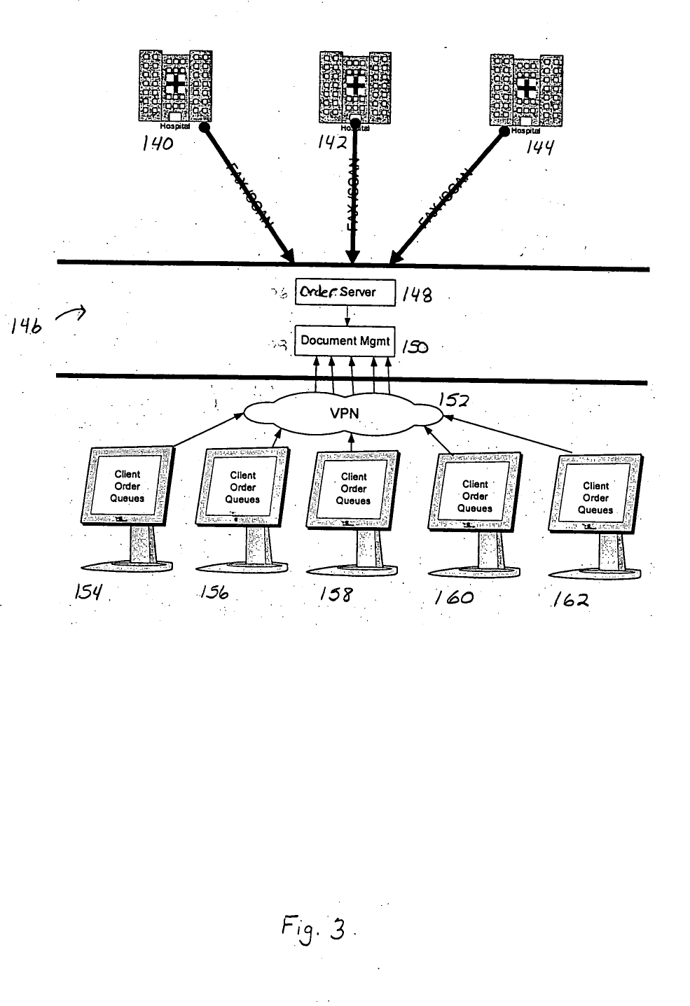

Referring to FIG. 3, a diagram of an order receiving server/document management server for an example embodiment of the present invention is shown. Orders are faxed, scanned, or emailed from hospital pharmacy information systems 140, 142, 144 and received at an order-receiving server 148 at a central order queue management site 146. The orders are digitized at the server 148 to create order images and transmitted to a document management server 150, which places orders in queues specific to each hospital. As indicated previously, the functions of an order receiving server and document management server may be combined in a single server referred to as an “order server.”

Pharmacists at a plurality of remote order processing centers 154, 156, 158, 160, 162 around the country connect via a virtual private network (VPN) 152 to the document management server 150 at the central order queuing site 146. Once connected to the document management server 150, they may view hospital order queues specific to each of the different hospitals they support. In an example embodiment of the present invention, each remote order processing center has the ability to view another remote order processing center's hospital queue as needed for disaster recovery in the event one remote order processing center experiences technical problems or is unable to provide order processing services.

Maintaining patient orders in a centralized server 148/document management server 150 results in additional benefits. Patient orders may be retrieved quickly for historical reference. In addition, they may be maintained in an active database according to state regulatory requirements. Finally, the centralized server/document management server serves as an active “failover” system for redundancy. It provides redundancy should one remote order processing center become non-functional. Through the centralized server 148/document management server 150 one remote order processing center may seamlessly access the order queue of any other hospital within minutes by electronic redirection of a hospital's queue to an alternative remote order processing center without requiring the hospital to change any of its processes. This redundancy ensures a consistent level of care to the hospitals. Additional redundancy is also maintained by the implementation of a backup order processing system running parallel with the “live” system. In the event of a failure with the “live” system, the backup solution engages automatically within minutes without any apparent interruption in service to the remote order processing centers and their hospitals.

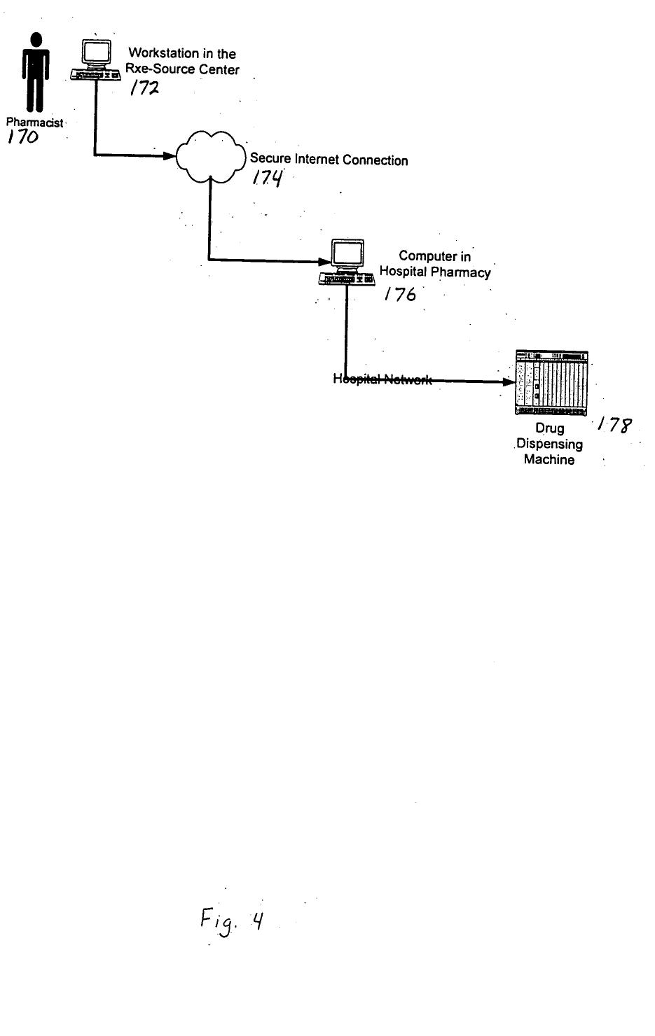

Referring to FIG. 4, a process for remote authorization of an automated medication dispensing system for an example embodiment of the present invention is shown. Using the present invention, medications may be remotely authorized in a hospital's pharmacy information system and in turn released from the automated medication dispensing system upon request by a healthcare provider. A pharmacist 170 receives an order for medication at a remote order processing center workstation 172. The pharmacist 170 remotely connects (via a secure Internet connection 174) the workstation 172 to a pharmacy information system computer at the hospital 176 from which the order was received. The pharmacist reviews, enters, and authorizes the order on the hospital's pharmacy information system. The hospital pharmacy information system 176 is interfaced to the automated medication dispensing system 178. Orders that are entered and authorized through the hospital pharmacy information system 176 appear in the automated medication dispensing system 178 so that the medication may be released to a healthcare provider who administers it to a patient.

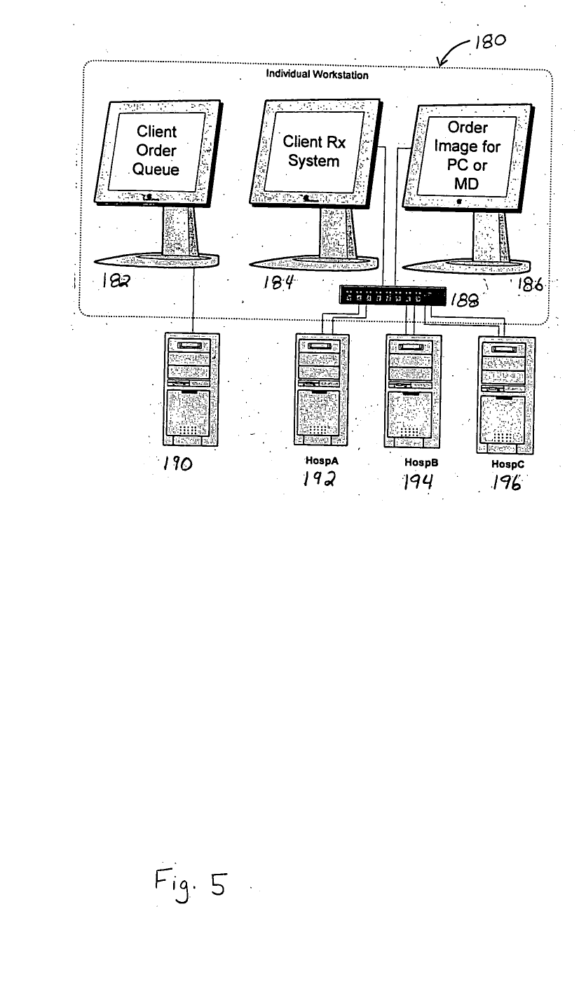

Referring to FIG. 5, a remote order processing standard station configuration for an example embodiment of the present invention is shown. Using this computer configuration, personnel at a remote order processing center may access multiple hospital pharmacy information systems 192, 194, 196 from one “station” 180. A station 180 consists of multiple monitors 184, 186 for accessing multiple hospital pharmacy information systems 192, 194, 196 during a remote order processing shift. The first monitor 182 displays the consolidated status of all hospital orders as well as specific order images from a computer accessing the order server 190. The second monitor 184 displays the hospital's pharmacy information system. The third monitor displays the order images if a hospital has an internal order management system such as PyxisConnect® from Pyxis Corporation or MedDirect™ from McKesson Automation, Inc. A data switchbox 188 allows-multiple computers to share a single monitor, keyboard, and mouse for saving space at the workstation 180.

The automatic display of a corresponding hospital's pharmacy information system on an alternate monitor 184 is a key feature of the invention that ensures remote order processing personnel enter the correct patient's order into the correct hospital's pharmacy information system as hospital “order queues” are selected from a screen that shows details of each order queue for each hospital. This feature helps to ensure patient safety and accuracy.

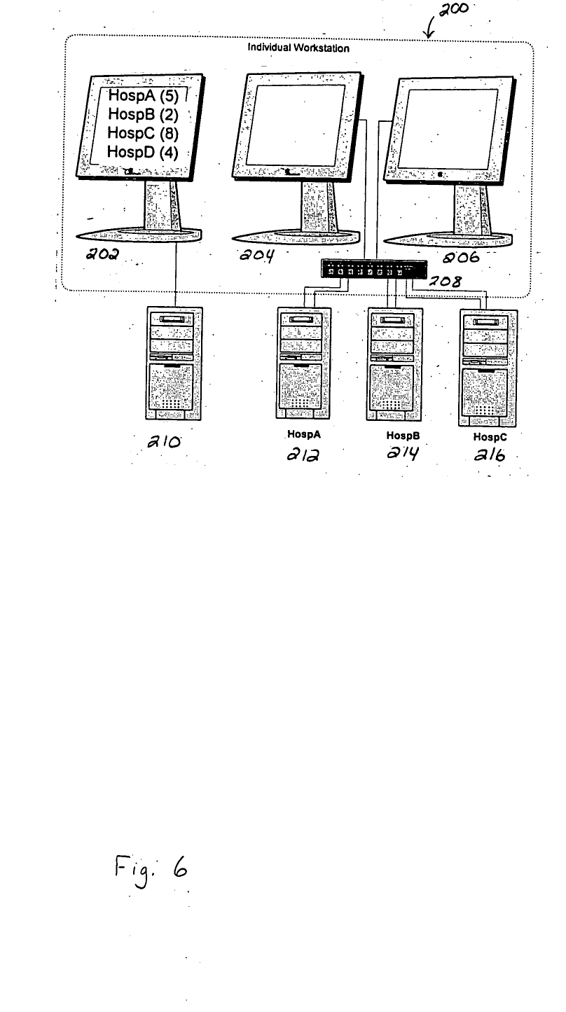

Referring to FIG. 6, a remote order processing station workflow for viewing a consolidated queue for an example embodiment of the present invention is shown. Remote order processing personnel may use this station 200 to view and access the hospital order queues. The consolidated queue displays orders for the hospitals supported by the remote order-processing center. The first monitor 202 displays the consolidated status of all hospital orders with the maximum time any order sheet has remained in the queue. The order queues are updated in real time as orders are received from the hospitals at the order server 210. Additional monitors 204, 206 are used for access to hospital pharmacy information systems 212, 214, 216.

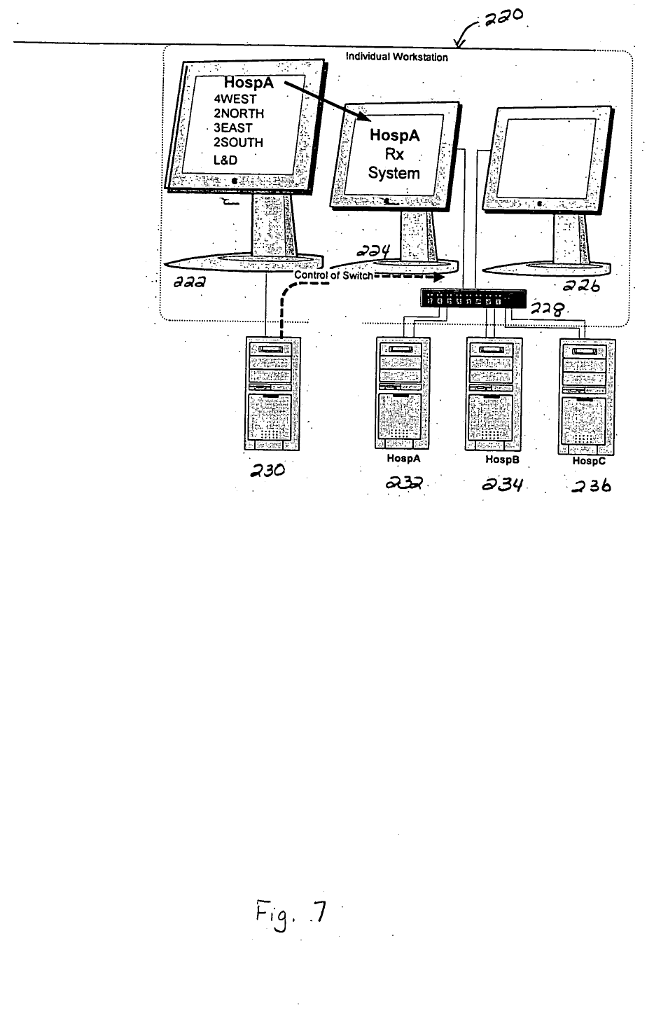

Referring to FIG. 7, a remote order processing station workflow for accessing specific hospital orders for an example embodiment of the present invention is shown. Remote order processing personnel may use this station 220 to select a specific hospital from the consolidated queue to view orders and access the hospital's pharmacy information system. When a hospital is selected, the first monitor 222 displays the list of selected hospital specific orders in the queue. When the selection is made, an automated command is sent to a computer 228 to automatically display the appropriate hospital's pharmacy information system on the second monitor 224. For example, if HospA is selected, the hospital pharmacy information system for HospA 232 is accessed. Similarly, the hospital pharmacy information systems for other hospitals 234, 236 may be accessed automatically when selected by a user.

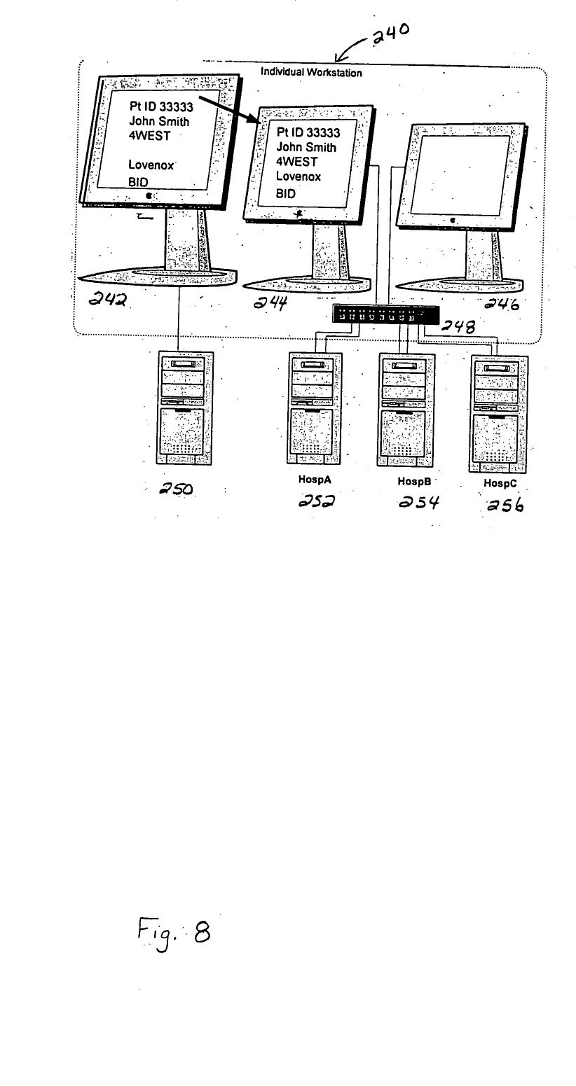

Referring to FIG. 8, a remote order processing station workflow for accessing a specific order for an example embodiment of the present invention is shown. Remote order processing personnel may use this station 240 to view a specific order from the hospital specific order queue and enter the order into the hospital's pharmacy information system. The first monitor 242 displays an image of the order selected by the user. After reviewing the order, the user may enter it into the hospital's pharmacy information system 244.

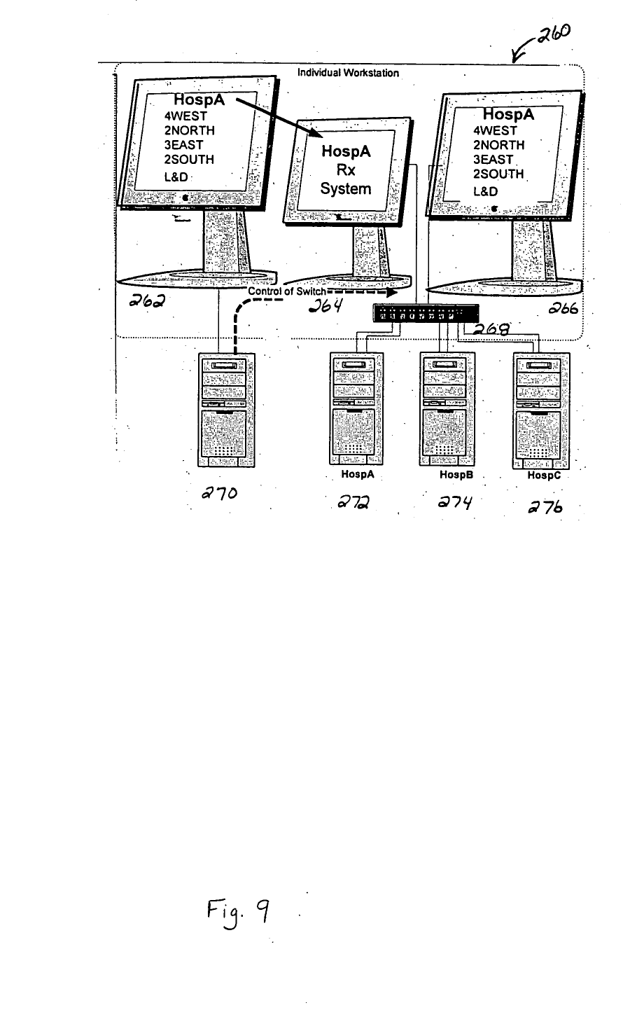

Referring to FIG. 9, a remote order processing station workflow for accessing specific hospital orders for hospitals with internal order management systems for an example embodiment of the present invention is shown. Remote order processing personnel may use this station 260 to view orders for hospitals with internal order management systems such as PyxisConnect and MedDirect. Orders for hospitals with this technology remain at the hospital and are accessed remotely from remote order processing centers to be processed.

After a user selects a hospital, the first monitor 262 displays a list of hospital specific orders in the queue. When the selection is made, an automated command is sent to a computer 268 to automatically display the appropriate hospital's pharmacy information system on the second monitor 264. For example, if HospA is selected, the hospital pharmacy information system for HospA 272 is accessed. Similarly, the hospital pharmacy information systems for other hospitals 274, 276 may be accessed automatically when selected by a user.

When the hospital pharmacy information system appears on the second monitor 264, the hospital's internal order management queue appears on the third monitor 266. Order images may then be accessed directly by the pharmacist from the hospital's internal order management system.

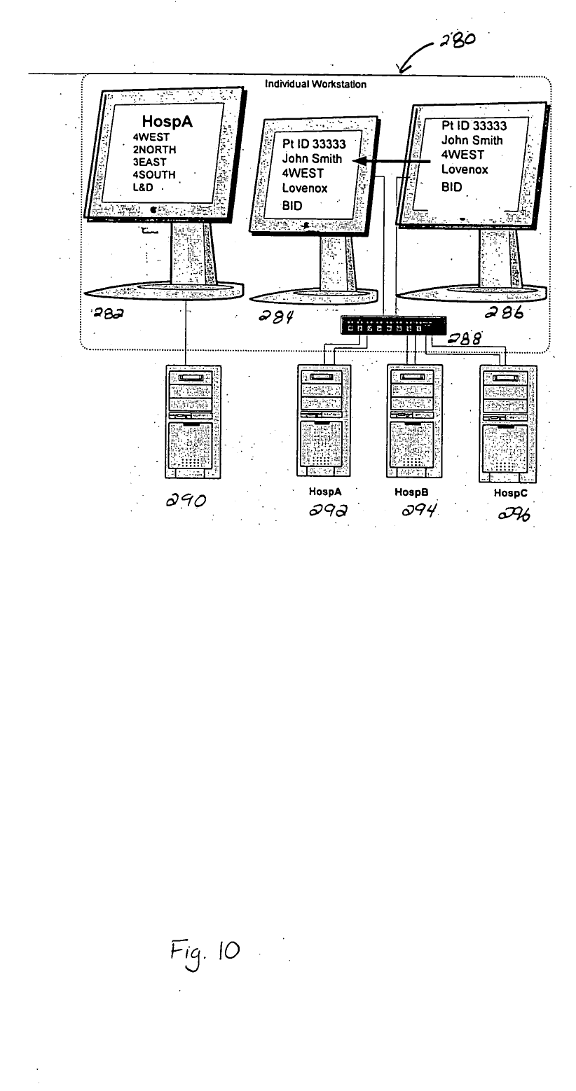

Referring to FIG. 10, a remote order processing station workflow for entering hospital orders using a hospital internal order management system for an example embodiment of the present invention is shown. Remote order processing personnel may use this station 280 to view and enter orders for hospitals with internal order management systems such as PyxisConnect and MedDirect. The remote order processing consolidated hospital order queue receives the number of orders in each queue for each hospital with an internal order management system. However, order images are displayed from another computer on another monitor because remote order processing personnel process the orders directly on the internal order management system at the hospital. To facilitate workflow, the order queue information is retained in the consolidated queue so that remote order processing personnel may indicate when orders are completed even though they are viewing the orders in the hospital's internal order management system.

After a user selects a hospital, the first monitor 282 displays a list of hospital specific orders in the queue. The second monitor 284 displays the hospital pharmacy information system in which the order is entered. The third monitor 286 displays the actual order image to be entered on the hospital's pharmacy information system. The order is then linked to the internal order management system located at the hospital site.

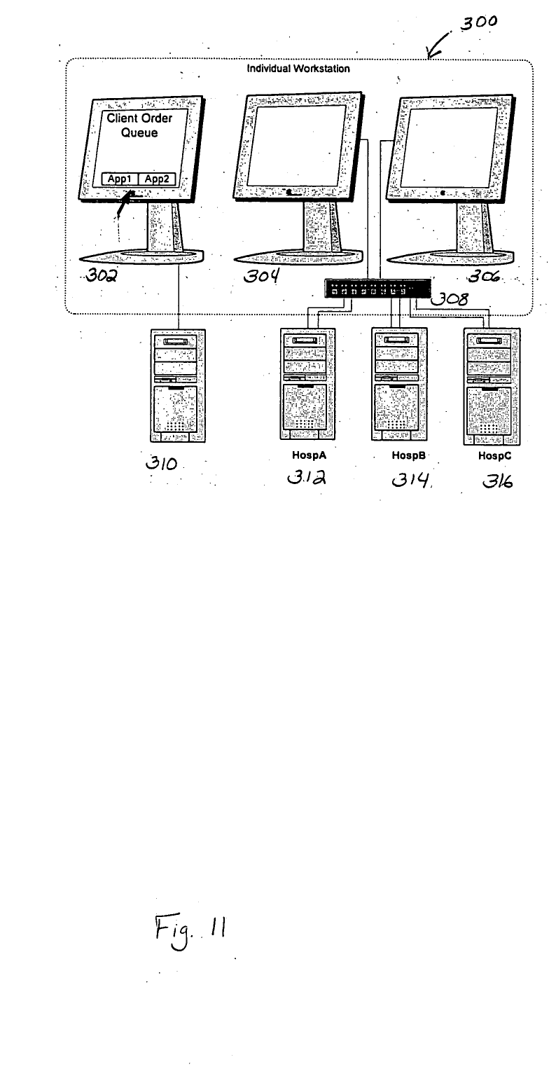

Referring to FIG. 11, a description of remote order processing proprietary software applications for an example embodiment of the present invention is provided. A plurality of remote order processing applications at a workstation 300 may be accessed from the same computer 310 as the consolidated queue information. The first monitor 302 displays the consolidated status of all hospital orders as well as specific order images. In addition, the first monitor 302 may display a remote order processing center's proprietary clinical intervention automated tracking application (App1) where order consultations are documented and reported back to the hospital. Consultations are defined as any additional action that must be taken to ensure the correct medication is entered for the patient including clinical interventions, clarifications, and automated medication dispensing cabinet concerns. Consultations are tracked and reported back to the hospitals by the remote order processing personnel or by the hospitals actively accessing their reports via a portal over the Internet. Hospitals may obtain consultation detail, volume, and statistical reports as frequently as desired. Copies of the original order sheets are included with some consultation reports as additional documentation for the hospital pharmacy. The clinical intervention application remains minimized at the bottom of the screen until needed. Finally, an application (App2) for accessing a hospital policy data repository may also be displayed from this monitor 302 to provide to remote order processing center personnel a quick reference to teach the pharmacist about the hospital's specific policies regarding clinical initiatives, pharmacy information system contacts, etc. The centralized data repository maintains all hospital specific clinical initiatives and policies and is accessed from the same VPN connection as the order processing-system. It may be used to quickly search and confirm hospital policy information during order entry to ensure hospital policy and safety initiatives are maintained. The clinical initiatives of the clinical intervention application may be configured according to the hospital's policies to ensure order processing compliance. These features provide for automated due diligence by allowing for quick implementation and quick retrieval of hospital policies and clinical initiatives.

Hospital queues are displayed for each remote order processing center according to the hospitals each center supports. Several views of each hospital are provided to facilitate order processing by pharmacy personnel.

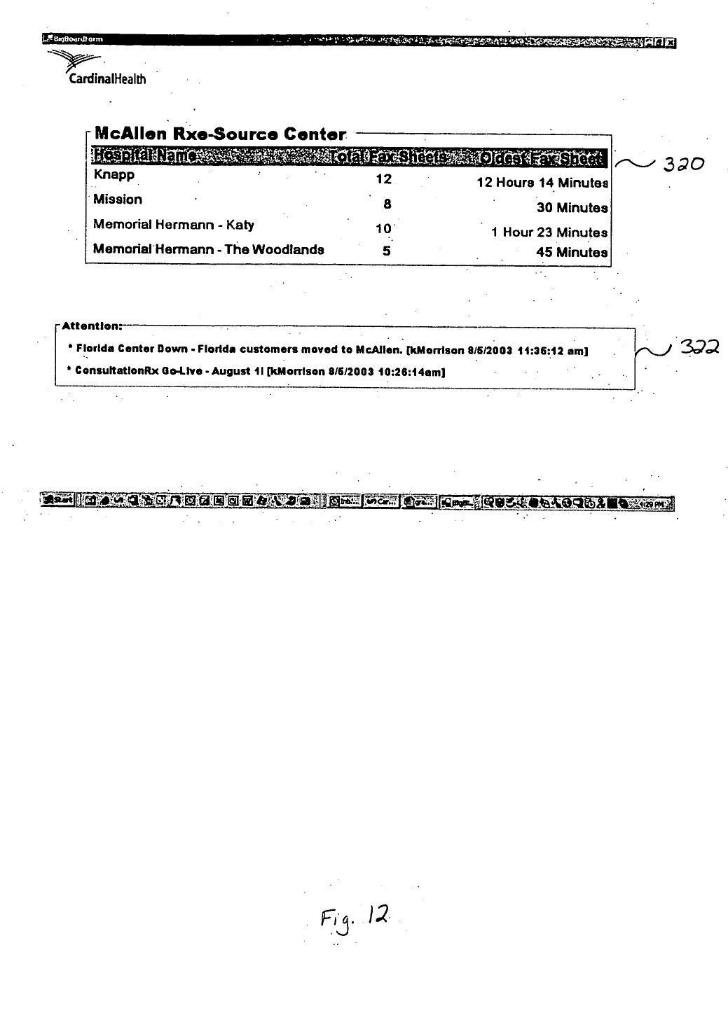

Master Hospital Queue (Big Board): In an example embodiment of the present invention, each remote order processing center is equipped with a wall-mounted plasma screen for viewing a master hospital queue. Referring to FIG. 12, a master hospital queue screen for an example embodiment of the present invention is shown. The master hospital queue view 320 lists on the plasma screen for each hospital the total number of orders in the hospital queue and the time of the oldest order in the queue. This view is coded so that hospital names are distinguished (e.g., displayed in different colors or different fonts) to bring attention to hospital queues that have orders that are classified as STAT (emergency), orders that are close to breaching the hospital's service level agreement (SLA warning), and orders that have breached the hospital's service level agreement (SLA breach). In addition, this view contains a message board 322 to aid in communication between a corporate office and the remote centers. Important information that needs to be viewed by all remote order processing center personnel may be sent to the centers by administrators at the corporate office.

The following tables illustrate alternative flows that may occur.

TABLE 1
Typical Flow - Master Queue

| Seq. | Actor | Description |
|---|---|---|
| 1 | User | Logs into the Big Board server using a user |
| | | identifier and password that pertains to a |
| | | specific center. |
| 2 | System | Displays the Hospital Name, Number of Fax |
| | | Pages, and the Age of the Oldest Fax Page |
| | | for all the hospitals in the master queue |
| | | that have at least one fax page and that |
| | | are associated with a particular center. |
| | | The orders are configurable by center. |

TABLE 2
Alternate Flow #1 - SLA Breach

| Seq. | Actor | Description |
|---|---|---|
| 1 | Center | Has a hospital that breaches a Service |
| | | Level Agreement. |
| 2 | System | Displays this hospital name in red if |
| | | there is not also a STAT order for that |
| | | hospital. |

TABLE 3
Alternate Flow #2 - SLA Warning

| Seq. | Actor | Description |
|---|---|---|
| 1 | Center | Has a hospital with a warning threshold |
| | | of breaching a Service Level Agreement. |
| 2 | System | Displays this hospital name in yellow if |
| | | there is not also a STAT order or an |
| | | SLA breach. |

TABLE 4
Alternate Flow #3 - STAT

| Seq. | Actor | Description |
|---|---|---|
| 1 | Center | Has a hospital with a Stat page. |
| 2 | System | Displays this hospital name at the top |
| | | of the list in green. |

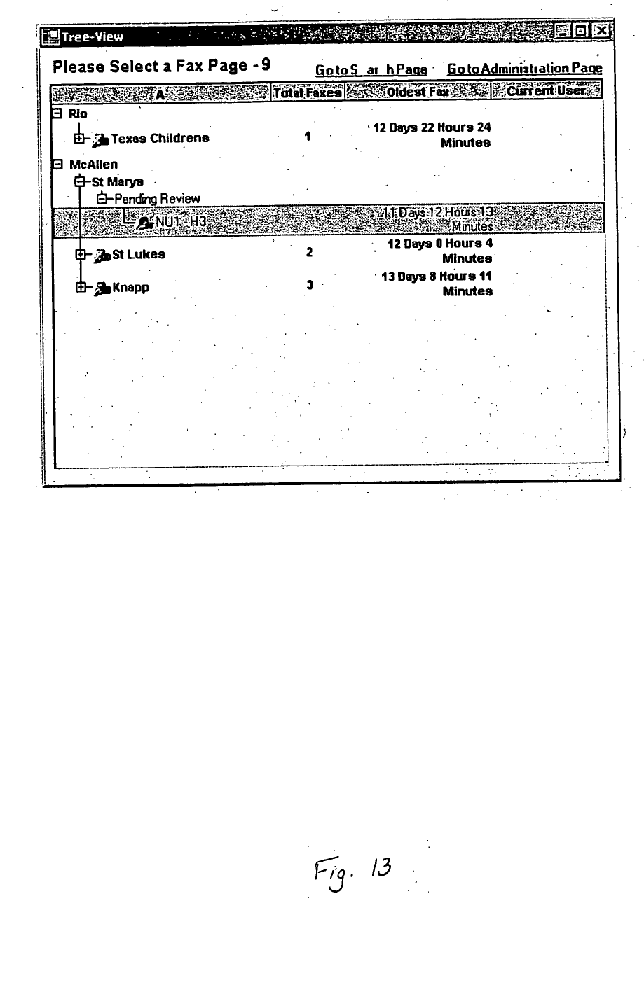

Hospital Detail Queue (Treeview): Referring to FIG. 13, a hospital detail queue screen for an example embodiment of the present invention is shown. This view lists each hospital on the computer screen in an expanded view or “treeview” with the same information as the master hospital queue in addition to information informing the user if someone is already processing the order image. This view further organizes each hospital queue into three sub-queues: New, On-Hold, and Pending Rph Validation where the order sheets may be accessed for processing. Order sheets are titled with the nursing unit location from which they were sent. Further, remote order processing personnel may route order sheets to other queues established for each hospital in addition to other hospital queues in the event an order sheet appears in an incorrect hospital queue. As with the master hospital queue, orders in this queue are distinguished (e.g., color coded) to inform users of STAT orders, orders about to breach a service level agreement, and orders that have already breached a service level agreement.

The following tables illustrate alternative flows that may occur.

TABLE 5
Typical Flow

| Seq. | Act r | Description |
|---|---|---|
| 1 | User | Logs into the Tree-View application. |
| 2 | System | Displays the center name, the length of time the |
| | | oldest order has been in the queue, and the total |
| | | orders in the queue for each center that the user |
| | | has rights to view sorted alphabetically by center |
| | | name. The center will not show up on the Tree-View |
| | | if it has no fax pages to display. |
| 3 | User | Expands a particular center. |
| 4 | System | Removes the oldest order and total order information |
| | | from the center node that has been expanded and |
| | | displays the all of the hospitals in the center |
| | | sorted alphabetically, along with the oldest order |
| | | and total orders for each of the hospitals. |
| | | The hospital will not show up on the Tree-View if |
| | | it has no fax pages to display. |
| 5 | User | Expands a particular hospital. |
| 4 | System | Removes the oldest order and total order information |
| | | from the hospital node that has been expanded and |
| | | displays the queue name, oldest order and total |
| | | orders for each of the queue categories that have |
| | | fax pages. The queue will not show up on the |
| | | Tree-View if it has no fax pages to display. |
| 5 | User | Expands one of the queues. |
| 6 | System | Removes the oldest order and total order information |
| | | from the queue node that has been expanded and |
| | | displays the Nursing Unit fax pages and the amount |
| | | of time each fax page has been in the queue sorted |
| | | oldest to newest. |
| 7 | User | Double-clicks on the fax page to edit. |
| 8 | System | Removes the Tree-View from the screen and displays |
| | | the Fax-View page. |

TABLE 6
Alternate Flow #1: Unknown Fax

| Seq. | Actor | Description |
|---|---|---|
| 1 | User | Notices a fax came in with an unknown hospital name. |
| 2 | User | Right clicks on the unknown hospital. |
| 3 | System | Displays a list of hospitals that get faxes sent to the |
| | | center. |
| 4 | User | Clicks on the hospital that the unknown fax belongs to. |
| 5 | System | Saves the hospital name chosen as the hospital name and |
| | | displays the fax on the Tree-View accordingly. |

TABLE 7
Alternate Flow #2 - SLA Breach

| Seq. | Actor | Description |
|---|---|---|
| 1 | Center | Has a fax page that has breached an SLA. |
| 2 | System | Displays a red pin next to the hospital if there |
| | | is not also a STAT order for that hospital. |
| 3 | User | Expands the hospital that has a fax page that |
| | | breached SLA. |
| 4 | System | Displays a red pin next to the queue that contains |
| | | the SLA Breach if there is not also a STAT order |
| | | in that particular queue. The Hospital name will no |
| | | longer have a red pin next to it. |
| 5 | User | Expands the queue that has a fax page that |
| | | breached SLA. |
| 6 | System | Displays a red pin next to the fax page that |
| | | breached SLA. The Hospital name and queue will no |
| | | longer have a red pin next to it. |

TABLE 8
Alternate Flow #3 - SLA Warning

| Seq. | Actor | Description |
|---|---|---|
| 1 | Center | Has a fax page that comes within its warning |
| | | threshold of breaching an SLA. |
| 2 | System | Displays a yellow pin next to the hospital name |
| | | if there is not also a STAT order or an SLA Breach |
| | | for that hospital. |
| 3 | User | Expands the hospital that has a fax page with an |
| | | SLA warning. |
| 4 | System | Displays a green pin next to the queue that |
| | | contains the SLA Warning if there is not also |
| | | a STAT order or an SLA Breach in that particular |
| | | queue. The Hospital name will no longer have a |
| | | green pin next to it. |
| 5 | User | Expands the queue that has a fax page with an |
| | | SLA warning. |
| 6 | System | Displays a green pin next to the fax page with |
| | | the SLA Warning. The Hospital name and queue |
| | | will no longer have a green pin next to it. |

TABLE 9
Alternate Flow #4 - STAT Order

| Seq. | Actor | Description |
|---|---|---|
| 1 | User | Has a fax page that is a STAT. |
| 2 | System | Displays a green pin next to the hospital name. |
| 3 | User | Expands the hospital that has a STAT fax page. |
| 4 | System | Displays a blue pin next to the queue that contains the |
| | | STAT. The Hospital name will no longer have a blue pin |
| | | next to it. |
| 5 | User | Expands the queue that has a STAT fax page. |
| 6 | System | Displays a blue pin next to the STAT fax page. The |
| | | Hospital name and queue will no longer have a blue |
| | | pin next to it. |

TABLE 10
Alternate Flow #5 - Go to Search Page

| Seq. | Actor | Description |
|---|---|---|
| 1 | User | Clicks the ‘Go to Search Page’ link. |
| 2 | System | Minimizes the Tree-View and maximizes the Search |
| | | screen. |

TABLE 11
Alternate Flow #6 - Go to Administration Page

| Seq. | Actor | Description |
|---|---|---|
| 1 | User | Clicks the ‘Go to Administration Page’ link. |
| 2 | System | Minimizes the Tree-View and maximizes the |
| | | Administration screen. |

TABLE 12
Exception Flow #1 - Fax Page in use - Unlocking Page

| Seq. | Actor | Description |
|---|---|---|
| 1 | New | Clicks on a fax page that is being used by |
| | User | someone else. |
| 2 | System | Displays a message that says, “Another user |
| | | is currently editing the fax page, do you wish |
| | | to unlock it?” |
| 3 | New | Presses the ‘Yes’ button. |
| | User | |
| 4 | System | Displays the fax page with all fields and |
| | | buttons enabled. |
| 5 | New | Enters meta-data and presses the ‘Save & Next’ |
| | User | or ‘Save & Return’ button. |
| 6 | System | Saves information. |
| 7 | Old User | Presses ‘Save & Next’ or ‘Save & Return’ |
| | | in order to save the meta-data entered. |
| 8 | System | Displays an error message that says, ‘You cannot |
| | | save this information because it has been altered |
| | | by another user’. |

TABLE 13
Exception Flow #2 - Fax Page in use - Not Unlocking Page

| Seq. | Actor | Description |
|---|---|---|
| 1 | User | Clicks on a fax page that is being used by |
| | | someone else. |
| 2 | System | Displays a message that says, “Another user |
| | | is currently editing the fax page, do you wish |
| | | to unlock it?” |
| 3 | User | Presses the ‘No’ button. |
| 4 | System | Displays the fax page with the ‘Save & Next’ |
| | | and ‘Save & Return’ buttons disabled. |

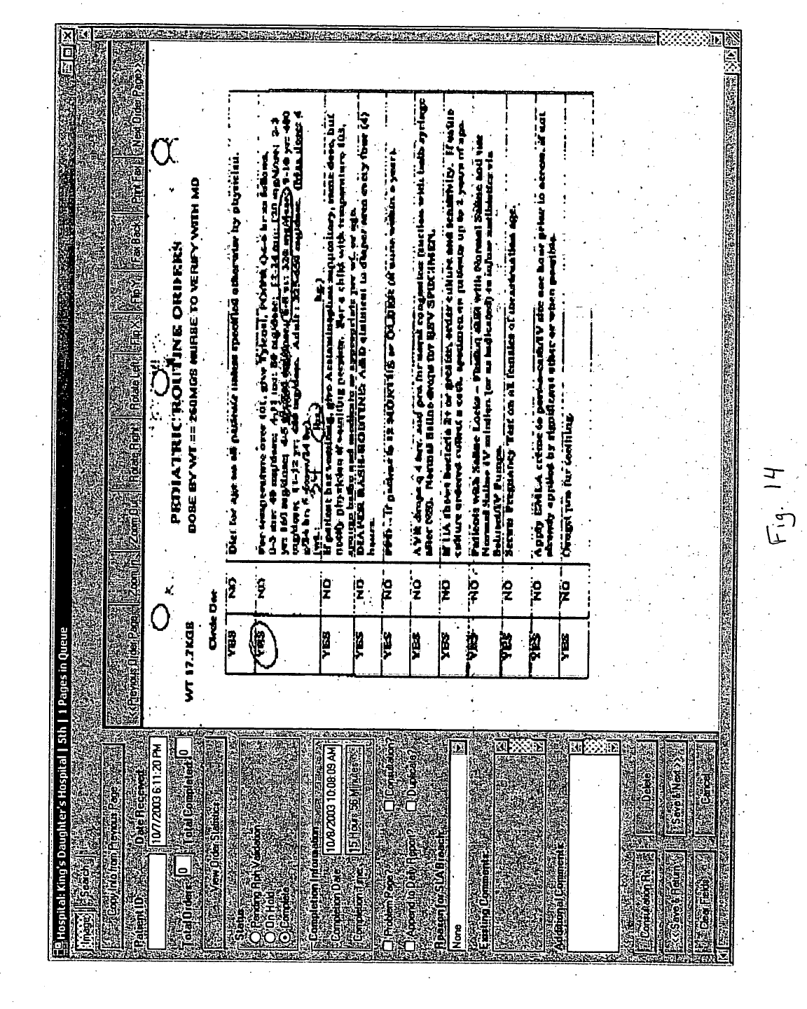

Order View Display: Referring to FIG. 14, an order view screen for an example embodiment of the present invention is shown. Once an order image is selected from the hospital queue, this view displays the electronic image of the order in addition to the following annotated fields: patient identifier, total orders on sheet, total orders completed, reasons for service level agreement breach (if applicable), status assignments/routing, checks to indicate if any of the orders were involved with a consultation, duplicate orders, problems with the page, and an indicator to indicate whether the order image should be appended to a daily consultation detail report as backup documentation for the hospital. Additional comment fields are also included as well as buttons to indicate the next screen that should appear once the order image is completed. Also, this screen provides remote order processing personnel with options to return order sheets to hospitals that request missing information or report sheets that are illegible, etc.

The following tables illustrate alternative flows that may occur.

TABLE 14
Typical Flow

| Seq. | Actor | Description |
|---|---|---|
| 1 | User | Clicks on a fax page in the Tree-View screen. |
| 2 | System | Checks to see if the next fax page is locked, and |
| | | locks page if applicable (see exceptions #2 and #3 |
| | | for details on the locking process). |
| 3 | System | Removes the Tree-View page from the screen and |
| | | displays the Fax-View page shown above populated |
| | | with data derived from the ContentEntry( ) method |
| | | in the RioConnector. |
| 4 | User | Enters meta-data information, selects a queue to |
| | | put the order into, and presses the ‘Save & Return’ |
| | | button. |
| 5 | System | Saves the meta-data information using the |
| | | SaveContent( ) method in the RioConnector, removes |
| | | the Fax-View from the screen, and displays an |
| | | updated Tree-View page. |

TABLE 15
Alternate Flow #1 - Save & Next

| Seq. | Actor | Descripti n |
|---|---|---|
| 1 | User | Enters meta-data information, selects a queue |
| | | to put the order into, and presses the |
| | | ‘Save & Next’ button. |
| 2 | System | Unlocks the current page, finds the node |
| | | containing the current content id in the |
| | | Tree-View, and gets the next node in the queue. |
| 3 | System | Checks to see if the next fax page is locked, |
| | | and locks page if applicable (see exceptions |
| | | #2 and #3 for details on the locking |
| | | process). |
| 4 | System | Saves the meta-data information for the current |
| | | content id using the SaveContent( ) method in |
| | | the RioConnector, and displays the meta- |
| | | data of the next fax page in the queue. |
| | | Will move to the first page in the queue if a |
| | | STAT comes in. |
| | | The user must choose the upper-most fax page they |
| | | wish to edit b/c Save & Next will not go back up |
| | | to the top of the queue unless there is a STAT page. |
| | | This button will be grayed out if accessed through |
| | | the Search Screen. |

TABLE 16
Alternate Flow #2 - Clear Fields

| Seq. | Actor | Description |
|---|---|---|
| 1 | User | Enters meta-data information, selects a queue |
| | | to put the order into, and presses the ‘Clear |
| | | Fields’ button. |
| 2 | System | Clears all of the meta-data fields and removes |
| | | any selections on the Fax-View screen. |

TABLE 17
Alternate Flow #3 - Cancel

| Seq. | Actor | Description |
|---|---|---|
| 1 | User | Enters meta-data information, selects a queue |
| | | to put the order into, and presses the |
| | | ‘Cancel’ button. |
| 2 | System | Unlocks the current page. |
| 2 | System | Removes the Fax-View page from the screen, and |
| | | displays the Tree-View page without saving the |
| | | meta-data that was entered. |

TABLE 18
Alternate Flow #4 - Copy Info from Previous Page

| Seq. | Actor | Description |
|---|---|---|
| 1 | User | Clicks the ‘Copy Info from Previous Page’ button. |
| | | This will only be enabled if there is previous infor- |
| | | mation to append. |
| | | There will not be previous information to append if the |
| | | fax page is directly selected from the Tree-View. |
| 2 | System | Copies the patient identifier entered for the previous |
| | | fax page and pastes it into the current fax page. |

TABLE 19
Alternate Flow #5 - Launch ConsultationRx

| Seq. | Actor | Description |
|---|---|---|
| 1 | User | Presses the Launch ConsultationRx button. |
| 2 | System | If there is a window handle, the application |
| | | maximizes the instance of Consultation Rx that |
| | | is already open and minimizes the Fax-View. |
| | | If there is not a window handle, the application |
| | | launches ConsultationRx and minimizes the Fax-View. |

TABLE 20
Alternate Flow #6 - Print

| Seq. | Actor | Description |
|---|---|---|
| 1 | User | Presses the Print button. |
| 2 | System | Prints only the image to the local fax machine |
| | | or network printer at the center - there will |
| | | be no printer dialog displayed. |

TABLE 21
Alternate Flow #7 - Fax Back

| Seq. | Actor | Description |
|---|---|---|
| 1 | User | Presses the Fax Back button. |
| 2 | System | Displays a printer dialog. |
| 3 | User | Enters the fax number he/she wishes to fax the image to. |
| 4 | System | Faxes the image to the specified location. |

TABLE 22
Alternate Flow #8 - Zoom

| Seq. | Actor | Description |
|---|---|---|
| 1 | User | Presses the Zoom In button. |
| 2 | System | Displays the original image size * 2, and |
| | | scrollbars will automatically appear |
| | | if the image becomes larger than the panel |
| | | the picture resides on. |
| 3 | User | Presses the Zoom Out button. |
| 4 | System | Displays the original image size/2. |

TABLE 23
Alternate Flow #9 - Unknown Hospital

| Seq. | Actor | Description |
|---|---|---|
| 1 | User | Clicks on a fax page that resides in an |
| | | unknown hospital. |
| 2 | System | Uses the Center id passed into the Fax-View |
| | | to retrieve all of the hospitals in that |
| | | particular center. |
| 3 | System | Displays the hospitals in a drop-down list, |
| | | and displays ‘Unknown’ as the hospital |
| | | name at the top of the form. |
| 4 | User | Selects the hospital that the unknown fax |
| | | belongs to from the drop-down list, enters |
| | | all other applicable meta-data, and presses |
| | | one of the Save buttons. |
| 5 | System | Validates that a hospital was selected, and |
| | | uses the SaveContent( ) method to save |
| | | all the meta-data. |

TABLE 24
Alternate Flow #9 - Consultations

| Seq. | Actor | Description |
|---|---|---|
| 1 | User | Clicks on a fax page in the Search screen. |
| 2 | System | Disables the ‘Save & Next’, ‘Next’, and |
| | | ‘Previous’ buttons on the Fax-View. |
| 3 | User | Presses the ‘Save & Return’ button. |
| 4 | System | Maximizes the Search page and minimizes the |
| | | Fax-View. |

TABLE 25
Alternate Flow #10 - Next

| Seq. | Actor | Description |
|---|---|---|
| 1 | User | Clicks the Next button at the top of the |
| | | Fax-View page. |
| 2 | System | Unlocks the current page, finds the node |
| | | containing the current content id in |
| | | the Tree-View, and gets the next node |
| | | in the queue. |
| 3 | System | Checks to see if the next fax page is |
| | | locked, and locks page if applicable |
| | | (see exceptions #2 and #3 for |
| | | details on the locking process). |
| 4 | System | Displays the meta-data of the next fax |
| | | page in the queue. |
| | | Will move to the first page in the queue |
| | | if a STAT comes in. |
| | | The user must choose the upper-most fax |
| | | page they wish to edit b/c Next will not |
| | | go back up to the top of the queue unless |
| | | there is a STAT page. |
| | | This button will be grayed out if accessed |
| | | through the Search Screen. |

TABLE 26
Alternate Flow #11 - Previous

| Seq. | Actor | Description |
|---|---|---|
| 1 | User | Clicks the Previous button at the top of |
| | | the Fax-View page. |
| 2 | System | Unlocks the current page, finds the node |
| | | containing the current content id in |
| | | the Tree-View, and gets the previous node |
| | | in the queue. |
| 3 | System | Checks to see if the previous fax page is |
| | | locked, and locks page if applicable |
| | | (see exceptions #2 and #3 for details |
| | | on the locking process). |
| 4 | System | Displays the meta-data of the previous fax |
| | | page in the queue |
| | | Will move to the first page in the queue if |
| | | a STAT comes in. |
| | | The ‘Previous’ button will not show |
| | | the fax-page that was just edited, it will |
| | | show the fax-page that precedes the current |
| | | fax page in the updated Tree-View. |
| | | This button will be grayed out if accessed |
| | | through the Search Screen. |

TABLE 27
Exception Flow #1 - Unreadable Fax

| Seq. | Actor | Description |
|---|---|---|
| 1 | User | Selects a fax on the Tree-View page that is |
| | | unreadable. |
| 2 | User | Enters 0 for Total Orders, checks the checkbox |
| | | that indicates that the page is a problem, |
| | | chooses complete as the destination queue, |
| | | and clicks either ‘Save & Next’ or |
| | | ‘Save & Return’. |
| 2 | System | Does not validate that the patient id was |
| | | entered, saves the information, and |
| | | either proceeds to the next page or returns |
| | | to the Tree-View depending on what button |
| | | is pressed. |

TABLE 28
Exception Flow #2 - Forwarding to
a Page in Progress - Unlocking Page

| Seq. | Actor | Description |
|---|---|---|
| 1 | New | Enters meta-data information, selects a queue |
| | User | to put the order into, and presses the |
| | | ‘Save & Next’ button to get to the next |
| | | page of the fax which is currently being |
| | | modified by another user. |
| 2 | System | Displays a message box that says ‘This page |
| | | is currently being used by another |
| | | person, would you like to unlock the page?’ |
| 3 | New | Presses the ‘Yes’ button. |
| | User | |
| 4 | System | Displays the page with all fields and |
| | | buttons enabled. |
| 5 | New | Enters metadeta and presses the |
| | User | ‘Save & Next’ or the ‘Save & Return’ |
| | | button. |
| 6 | System | Saves meta-data. |
| 5 | Old User | Enters meta-data and presses the |
| | | ‘Save & Next’ or ‘Save & Return’ |
| | | button. |
| 6 | System | Displays an error message that says, ‘You |
| | | cannot save this information because it has |
| | | been altered by another user’. |

TABLE 29
Exception Flow #3 - Forwarding to a
Page in Progress - Not Unlocking Page

| Seq. | Actor | Description |
|---|---|---|
| 1 | New | Enters meta-data information, selects a queue |
| | User | to put the order into, and presses the |
| | | ‘Save & Next’ button to get to the next |
| | | page of the fax which is currently being |
| | | modified by another user. |
| 2 | System | Displays a message box that says ‘This page |
| | | is currently being used by another |
| | | person, would you like to unlock the page?’ |
| 3 | New | Presses the ‘No’ button. |
| | User | |
| 4 | System | Displays the page with the ‘Save & Next’ |
| | | and ‘Save & Return’ buttons disabled. |

TABLE 30
Exception Flow #4 - No Next Page

| Seq. | Actor | Description |
|---|---|---|
| 1 | User | Presses the ‘Save & Next’ or ‘Next’ |
| | | button in the Fax-View page when there are |
| | | no more fax pages for a particular queue |
| | | (ex: there are no more fax pages in |
| | | the New queue of Knapp hospital). |
| 2 | System | Removes the Fax-View page from the screen and |
| | | displays the Tree-View page. |

TABLE 31
Exception Flow #5 - No Previous Page

| Seq. | Actor | Description |
|---|---|---|
| 1 | User | Presses the ‘Previous’ button in the Fax-View |
| | | page when there are no previous fax pages to view |
| | | because the page was selected directly from the |
| | | Tree-View. |
| 2 | System | Grays out the Previous button. |

The centralized pharmacy order queuing and document management features of the present invention are used to route pharmacy orders to remote order processing center personnel who review, annotate, index, enter, and authorize pharmacy orders for medication from multiple hospitals while accessing various different pharmacy information systems. The faxed, emailed, or scanned pharmacy orders are converted to an electronic format (e.g., electronic order image) and placed in hospital specific queues identifying them by hospital name and nursing unit. Further, when a pharmacist at a remote processing center selects a hospital queue to review pending electronic orders, the system automatically launches the selected hospital's pharmacy information system for order review and authorization. Monitoring services based on service level agreements ensure pharmacy orders are entered and authorized in hospital pharmacy information systems according to specific hospital agreement parameters. Further, the technology provides clinical consultation tracking and reporting for each hospital as well as video conferencing capabilities to provide remote clinical consulting to hospital's clinical personnel. Finally, hospital specific clinical initiatives and hospital policies are collected from the hospitals and entered in a central data repository via an Internet connection. Once entered into the data repository, remote order processing center personnel can quickly search on specific hospital clinical initiatives during order entry to ensure hospital policy compliance even when the hospital pharmacy is closed.

Although the present invention has been described in relation to order processing services that may be offered to hospitals, it is understood that the services may be provided to any healthcare facility that provides medications to patients such as long term care facilities, specialty healthcare clinics, etc. While example embodiments of the invention have been illustrated and described, various modifications and combinations can be made without departing from the spirit and scope of the invention. Modifications, combinations, and equivalents to the system and method of the present invention are intended to be covered and claimed.

1. A method for remote processing of pharmacy orders:
establishing at an order server a plurality of order queues for a plurality of healthcare facilities, each of said order queues associated with one of said plurality of healthcare facilities;
assigning each of said plurality of order queues to one of a plurality of remote processing centers;
receiving at said order server a plurality of orders from said plurality of healthcare facilities;
adding each of said plurality of orders to one of said plurality of order queues associated with one of said plurality of healthcare facilities;
accessing one of said plurality of order queues from said one of said plurality of remote processing centers assigned to said order queue; and
processing said orders in said accessed order queue.

2. The method of claim 1 wherein processing said orders in said accessed order queue comprises accessing a pharmacy information system for said healthcare facility associated with said accessed order queue.

3. The method of claim 2 wherein accessing said pharmacy information system comprises automatically connecting to said pharmacy information system when said accessed order queue is accessed from said remote processing center assigned to said order queue.

4. The method of claim 2 further comprising dispensing a medication associated with an order in said accessed order queue from an automated medication dispensing system interfaced to said pharmacy information system.

5. The method of claim 1 wherein processing said orders in said accessed order queue comprises processing said orders according to service level commitments specified by said healthcare facility associated with said accessed order queue.

6. The method of claim 1 wherein processing said orders in said accessed order queue comprises processing said orders according to policies established by said healthcare facility associated with said accessed order queue.

7. A method for remote processing of pharmacy orders:
establishing at an order server a first order queue for a first healthcare facility;
establishing at said order server a second order queue for a second healthcare facility;
receiving at said order server a plurality of orders from said first healthcare facility;
receiving at said order server a plurality of orders from said second healthcare facility;
adding each of said plurality of orders from said first healthcare facility to said first order queue;
adding each of said plurality of orders from said second healthcare facility to said second order queue;
accessing orders from said first order queue and orders from said second order queue from a first remote processing center; and
processing at said first remote processing center said orders from said first order queue and orders from said second order queue.

8. The method of claim 7 further comprising accessing orders from said first order queue and orders from said second order queue from a second remote processing center upon failure of said first remote processing center to process orders.

9. The method of claim 7 wherein processing at said first remote processing center said orders from said first order queue comprises accessing a pharmacy information system for said first healthcare facility associated with said first order queue.

10. The method of claim 9 wherein accessing said pharmacy information system comprises automatically connecting to said pharmacy information system when said first order queue associated with said first healthcare facility is selected at said first remote processing center.

11. The method of claim 9 further comprising dispensing a medication associated with an order in said first order queue from an automated medication dispensing system interfaced to said pharmacy information system.

12. The method of claim 7 wherein processing at said first remote processing center said orders from said first order queue comprises processing said orders according to service level commitments specified by said first healthcare facility associated with said first order queue.

13. The method of claim 7 wherein processing at said first remote processing center said orders from said first order queue comprises processing said orders according to policies established by said first healthcare facility associated with said first order queue.

14. A system for remote processing of pharmacy orders comprising:
a plurality of order queues, each of said order queues associated with a healthcare facility;
an order server for receiving orders from said healthcare facilities and adding them to said order queues according to said associated healthcare facility and for responding to requests for accessing and processing orders in said plurality of order queues; and
at least one computer at least one remote processing center for accessing and processing orders in said plurality of order queues.

15. The system of claim 14 wherein said computer at said remote processing center is adapted to display a master healthcare facility queue view comprising the total number of orders in the healthcare facility queue and the time of the oldest order in the healthcare facility queue.

16. The system of claim 15 wherein said computer at said remote processing center is adapted to display a healthcare facility detail queue view comprising an expanded view of said healthcare facility queue and status information related to processing of an order in said healthcare facility queue.

17. The system of claim 16 wherein said status information relates to processing commitments specified in a service level agreement.

18. The system of claim 14 wherein said computer at said remote processing center is adapted to display an order view comprising an electronic image of an order from a selected healthcare facility queue.

19. The system of claim 14 further comprising a clinical intervention automated tracking application for documenting and reporting order consultations.

20. The system of claim 14 further comprising a second remote processing center for accessing and processing orders in said plurality of order queues when said at least one remote processing center fails to process orders.
